(19) United States
(12) Patent Application Publication
(10) Pub. No.: US 2006/0229274 A1
(43) Pub. Date: Oct. 12, 2006

(54) Weight reduction and inhibition of hypertriglyceridemia medicine containing ATP as effective ingredient

(76) Inventor: Chao Song Hsue, Taichung (CN)

Correspondence Address: BACON & THOMAS, PLLC, 625 SLATERS LANE, FOURTH FLOOR, ALEXANDRIA, VA, 22314, US

(21) Appl. No.: 11/452,912

(22) Filed: Jun. 15, 2006

Related U.S. Application Data
(63) Continuation-in-part of application No. 10/140,189, filed on May 8, 2002.

(51) Int. Cl. A61K 31/7076; A61K 31/522

(52) U.S. Cl. 514/47; 514/263.32

(57) ABSTRACT

A weight reduction and inhibition of Hypertriglyceridemia medicine contain ATP as an effective ingredient, and more particularly to one that takes advantage of a cyclic adenosine monophosphate (Cyclic AMP) produced from the decomposed ATP in human body to promote the increase of lipase, thus to activate the metabolism and decomposition of triglyceride in the human body.

### CROSS REFERENCES TO THE RELATED APPLICATIONS

This is a Continuation-in-part of U.S. application Ser. No. 10/140,189, filed May 8, 2002, now pending.

### BACKGROUND OF THE INVENTION

Description of Related Art

In a pharmacological sense, the working mechanism of the present invention is totally different from any weight reduction medicines, either National Health Department approved or USA FDA approved generally available in the market, there are three types of weight reduction medicines in the market, respectively:
1. P.P.A (Phenylpropanolamine), related to a sympathetic nerve stimulant essentially by affecting the hypothalamus nerve to create lack of appetite and frustrate the desire for food so as to cut down the amount of food taking, thus to achieve the purpose of weight reduction;
2. Xanical (Orlistat), related to an inhibitor for the lipase from the pancreas in the digestion duct by preventing fat in food from being decomposed and absorbed by the human body and thus to have the fat to be forthwith discharged out of the human body; furthermore, the oily excrement is common; and
3. Reductil (Subutramine), related to an inhibitor to the absorption of serum serotonin and noradrenaline, both conduction substances of the nerves system, functioning by developing the sense of SATIETY and thermogenesis.

WO01/28528 to Rapaport discloses and teaches methods for the chronic administration of adenosine, which are contrary to the acute delivery of the drug by injection or infusion. (See Abstract). Rapaport discloses and teaches an oral dosage form to be taken orally only. However, disodium salt or physiologically acceptable salt adenosine 5′-triphosphate(ATP) do not have an identical chemical structure as adenosine triphosphate(ATP). Disodium salt or physiologically acceptable salt adenosine 5′-triphosphate(ATP) also do not have an identical chemical structure as cyclic 3′,5′-AMP (cAMP). Rapaport teaches “Effective weight loss in humans can be either by desensitization the adipose tissue adenosine A1 receptors by themselves, or by desensitization in combination with adenosine antagonist such as caffeine or theophylline”, which are more effective in blocking the action of adenosine once its receptors became desensitized. However, Rapaport does not disclose, teach, suggest using ATP alone or using adenosine antagonist alone has little or unreliable weight loss or lipolytic effect. Rapaport does not disclose, teach, or suggest the requirement of co-existing of ATP and adenosine antagonist in terms of weight loss. Rapaport does not disclose, teach, or suggest the non-separatable relationship between ATP and adenosine antagonist in terms of weight loss. Rapaport does not disclose, teach, or suggest the necessary of ATP working together with adenosine antagonist in terms of weight loss. Rapaport does not disclose, teach, or suggest the use of cAMP. Rapaport does not disclose, teach, or suggest that ATP is a precursor; key element is cAMP in terms of weight loss. Rapaport does not disclose, teach, or suggest the use of theobromine, which is 3,7-dimethyl-xanthine. Rapaport does not disclose, teach, or suggest xanthines inhibit further degradation of ATP. Rapaport does not disclose, teach, or suggest when stable disodium salt ATP indigested into 5′-AMP has no lipolytic effect, which has no use in weight loss.

### SUMMARY OF THE INVENTION

The primary purpose of the present invention is to provide a weight reduction and inhibition of Hypertriglyceridemia medicine containing ATP to promote certain biologic enzymes, and thus encourages metabolism for weight reduction and breakdown of Triglyceride. To achieve this purpose, the present invention contains ATP as its effective ingredient and takes advantage of a cyclic Adenosine monophosphate (Cyclic AMP) produced from the decomposed ATP in the human body to promote the synthesis of lipase, thus activating the metabolism and decomposition of Triglyeride in the human body.

### BRIEF DESCRIPTION OF THE DRAWINGS

FIG. 1 is a view showing a chemical structure formula of ATP.

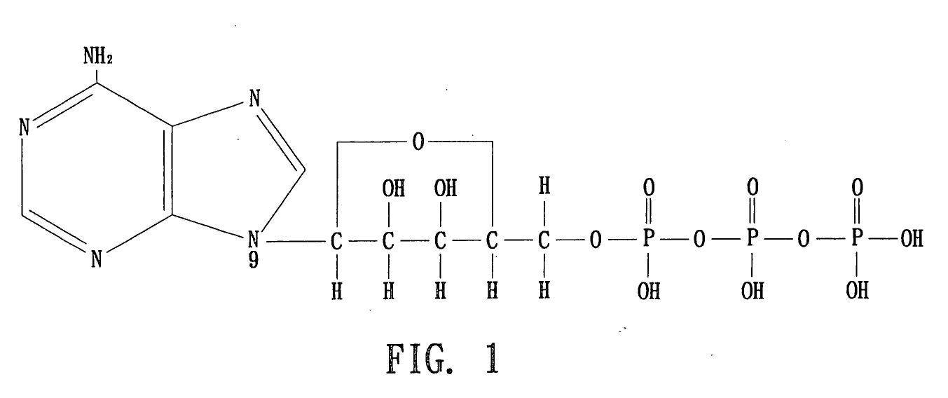

FIG. 2 is a view showing a chemical structure formula of Cyclic AMP.

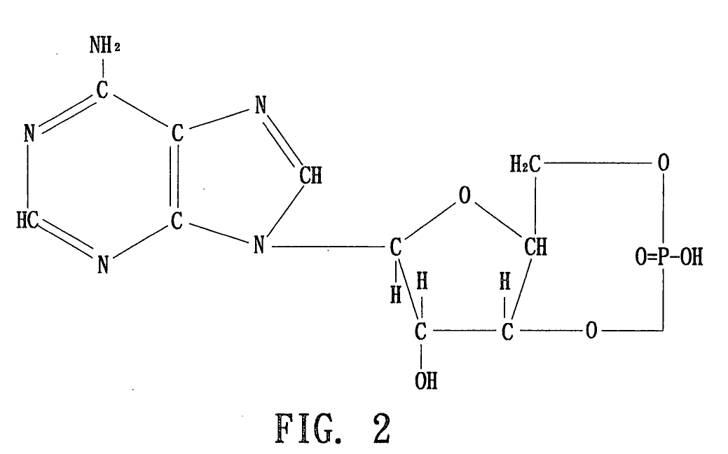

FIG. 3 is a schematic view showing biochemical reactions of the present invention.

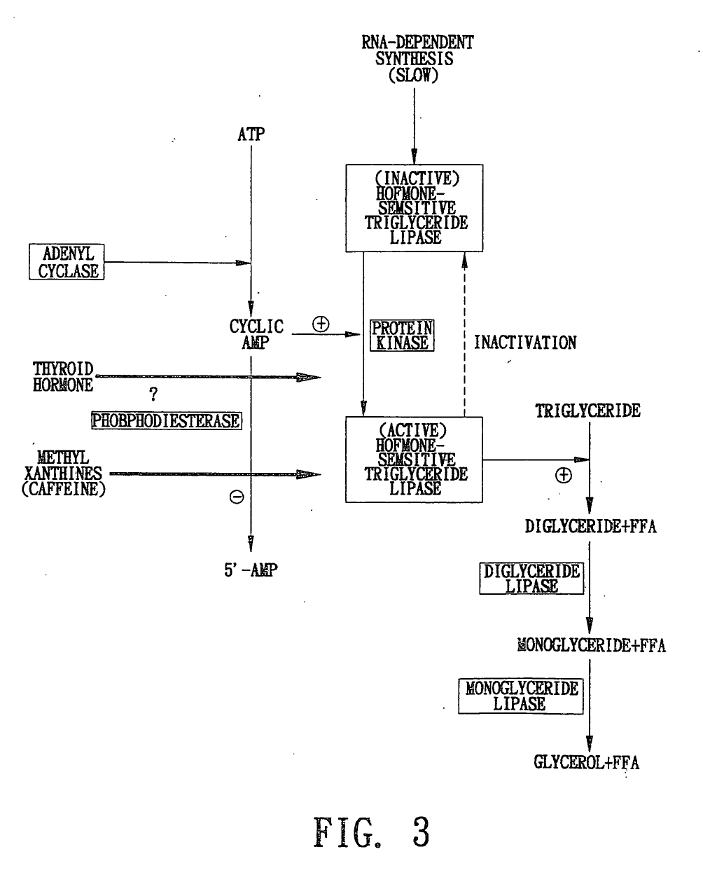

### DETAILED DESCRIPTION OF THE INVENTION

A weight reduction medicine and lysis of TG (Triglyceride) of the present invention contains ATP as its effective ingredient. ATP being subjected to reaction of adenyl clyase produces a cyclic AMP and the cyclic AMP in turn activates protein kinase to further encourage and activate lipase in the human body. Accordingly, FAT, adipose tissue and TG can break into diglyceride and free fatty acid (FFA), monoglyceride is then decomposed into glycrol and free fatty acid (FFA). As a result, TG is broken down into H₂O, CO₂ and energy. This not only provides the energy that the human body needs, but also achieves the purpose of weight reduction and the break down of TG.

The ATP of the present invention may be non-salt form dephosphorylatable ATP. The cyclic AMP of the present invention includes physiologically acceptable salts thereof.

It is to be noted that in the composition of the present invention, proper inhibitors may be added to maintain a certain transfer rate for ATP into Cyclic AMP so as to prevent subsequent reaction of the Cyclic AMP to adenosine-5′monophosphate. Those inhibitors are required to contain sufficient chemical properties to inhibit the Cyclic AMP from producing adenosine-5′-monophosphate. At least one from xanthine derivates may be selected as the inhibitor. If the xanthine derivatives are selected, any of a 1,3,7 trimethyl-xanthine, 1,3 dinethyl-xanthine or a 3,7 dimethyl-xanthine derivative or any of their derivatives may be options and any of the options are capable of providing inhibition results sufficient to maintain good transfer rate for ATP to produce the Cyclic AMP, thus to ensure of optimal activation for the lipase.

It should be noted that the composition itself and the components of the composition may be in a form that is suitable to be used externally and to be absorbed from skin, in a form that is suitable to be used nasally and to be absorbed from nasal mucous membrane, in a form that is suitable to be injected and to be absorbed intramuscularly, in a form that is suitable to be injected and to be absorbed intravenously, in a form that is suitable to be used rectally and to be absorbed from lower segment of intestinal tract, and in a form that is suitable to be taken orally and to be absorbed from gastrointestinal tract, as well as any other route of administration well known in the art.

As disclosed, the present invention achieves its purpose of weight reduction and decomposition of TG by promoting activation and increase of lipase to facilitate decomposition and metabolism of Triglyceride for removing fat accumulated in the human body, instead of inhibiting the normal operation of mechanism in the human body. The present invention is totally different than any of the prior art either in pharmacology or mechanical transfer, and creates comparatively less side effects to the human body.

What is claimed is:

1. A weight reduction and inhibition of hypertriglyceridemia composition comprising an effective amount of adenosine triphosphate (ATP) in a form that is suitable to be used externally and to be absorbed from skin.

2. The weight reduction and inhibition of hypertriglyceridemia composition comprising an effective amount of adenosine triphosphate (ATP) as defined claim 1, wherein said the effective amount of adenosine triphosphate (ATP) in a form that is suitable to be used nasally and to be absorbed from nasal mucous membrane.

3. The weight reduction and inhibition of hypertriglyceridemia composition comprising an effective amount of adenosine triphosphate (ATP) as defined claim 1, wherein said the effective amount of adenosine triphosphate (ATP) in a form that is suitable to be injected and to be absorbed intramuscularly.

4. The weight reduction and inhibition of hypertriglyceridemia composition comprising an effective amount of adenosine triphosphate (ATP) as defined claim 1, wherein said the effective amount of adenosine triphosphate (ATP) in a form that is suitable to be injected and to be absorbed intravenously.

5. The weight reduction and inhibition of hypertriglyceridemia composition comprising an effective amount of adenosine triphosphate (ATP) as defined claim 1, wherein said the effective amount of adenosine triphosphate (ATP) in a form that is suitable to be used rectally and to be absorbed from lower segment of intestinal tract.

6. A weight reduction and inhibition of hypertriglyceridemia composition comprising an effective amount of adenosine triphosphate (ATP) and an effective amount of at least one inhibitor selected from the group consisting of 1,3,7-trimethy-xantine, 1,3-dimethyl-xanthine and a 3,7-dimethyl-xanthine, wherein said the weight reduction and inhibition of hypertriglyceridemia composition comprising an effective amount of adenosine triphosphate (ATP) and an effective amount of at least one inhibitor selected from the group consisting of 1,3,7-trimethy-xantine, 1,3-dimethyl-xanthine and a 3,7-dimethyl-xanthine in a form that is suitable to be injected and to be absorbed intramuscularly.

7. The weight reduction and inhibition of hypertriglyceridemia composition comprising an effective amount of adenosine triphosphate (ATP) and an effective amount of at least one inhibitor selected from the group consisting of 1,3,7-trimethy-xantine, 1,3-dimethyl-xanthine and a 3,7-dimethyl-xanthine as defined claims 1 or 6, wherein said the adenosine triphosphate (ATP) and an effective amount of at least one inhibitor selected from the group consisting of 1,3,7-trimethy-xantine, 1,3-dimethyl-xanthine and a 3,7-dimethyl-xanthine in a form that is suitable to be used nasally and to be absorbed from nasal mucous membrane.

8. The weight reduction and inhibition of hypertriglyceridemia composition comprising an effective amount of adenosine triphosphate (ATP) and an effective amount of at least one inhibitor selected from the group consisting of 1,3,7-trimethy-xantine, 1,3-dimethyl-xanthine and a 3,7-dimethyl-xanthine as defined claims 1 or 6, wherein said the adenosine triphosphate (ATP) and an effective amount of at least one inhibitor selected from the group consisting of 1,3,7-trimethy-xantine, 1,3-dimethyl-xanthine and a 3,7-dimethyl-xanthine in a form that is suitable to be injected and to be absorbed intravenously.

9. The weight reduction and inhibition of hypertriglyceridemia composition comprising an effective amount of adenosine triphosphate (ATP) and an effective amount of at least one inhibitor selected from the group consisting of 1,3,7-trimethy-xantine, 1,3-dimethyl-xanthine as defined claims 1 or 6, wherein said the adenosine triphosphate (ATP) and an effective amount of at least one inhibitor selected from the group consisting of 1,3,7-trimethy-xantine, 1,3-dimethyl-xanthine and a 3,7-dimethyl-xanthine in a form that is suitable to be used externally and to be absorbed from skin.

10. The weight reduction and inhibition of hypertriglyceridemia composition comprising an effective amount of adenosine triphosphate (ATP) and an effective amount of at least one inhibitor selected from the group consisting of 1,3,7-trimethy-xantine, 1,3-dimethyl-xanthine and a 3,7-dimethyl-xanthine as defined claims 1 or 6, wherein said the adenosine triphosphate (ATP) and an effective amount of at least one inhibitor selected from the group consisting of 1,3,7-trimethy-xantine, 1,3-dimethyl-xanthine and a 3,7-dimethyl-xanthine in a form that is suitable to be used rectally and to be absorbed from lower segment of intestinal tract.

11. A weight reduction and inhibition of hypertriglyceridemia composition comprising an effective amount of non-salt form dephosphorylatable adenosine triphosphate (ATP), wherein said the weight reduction and inhibition of hypertriglyceridemia composition promotes activation and increase lipase to facilitate decomposition and metabolism of triglyceride for removing fat accumulated in a human body.

12. The weight reduction and inhibition of hypertriglyceridemia composition comprising an effective amount of non-salt form dephosphorylatable adenosine triphosphate (ATP) as defined claim 11, wherein said the weight reduction and inhibition of hypertriglyceridemia composition comprising an effective amount of non-salt form dephosphorylatable adenosine triphosphate (ATP) and an effective amount of at least one inhibitor selected from the group consisting of 1,3,7-trimethy-xantine, 1,3-dimethyl-xanthine and a 3,7-dimethyl-xanthine, so as to the weight reduction and inhibition of hypertriglyceridemia composition promotes activation and increase lipase to facilitate decomposition and metabolism of triglyceride for removing fat accumulated in a human body.

13. A weight reduction and inhibition of hypertriglyceridemia composition comprising an effective amount of cyclic AMP (cAMP) or physiologically acceptable salt thereof or both in a form that is suitable to be taken orally and to be absorbed from gastrointestinal tract.

14. The weight reduction and inhibition of hypertriglyceridemia composition comprising an effective amount of cyclic AMP (cAMP) or physiologically acceptable salt thereof or both and an effective amount of at least one inhibitor selected from the group consisting of 1,3,7-trimethy-xantine, 1,3-dimethyl-xanthine and a 3,7-dimethyl-xanthine as defined claim 13, wherein said the weight reduction and inhibition of hypertriglyceridemia composition comprising an effective amount of cyclic AMP (cAMP) or physiologically acceptable salt thereof or both and an effective amount of at least one inhibitor selected from the group consisting of 1,3,7-trimethy-xantine, 1,3-dimethyl-xanthine and a 3,7-dimethyl-xanthine in a form that is suitable to be taken orally and to be absorbed from gastrointestinal tract.

15. A weight reduction and inhibition of hypertriglyceridemia composition comprising an effective amount of cyclic AMP (cAMP) or physiologically acceptable salt thereof or both in a form that is suitable to be absorbed from skin.

16. The weight reduction and inhibition of hypertriglyceridemia composition comprising an effective amount of cyclic AMP (cAMP) or physiologically acceptable salt thereof or both and an effective amount of at least one inhibitor selected from the group consisting of 1,3,7-trimethy-xantine, 1,3-dimethyl-xanthine and a 3,7-dimethyl-xanthine as defined claim 15, wherein said the weight reduction and inhibition of hypertriglyceridemia composition comprising an effective amount of cyclic AMP (cAMP) or physiologically acceptable salt thereof or both and an effective amount of at least one inhibitor selected from the group consisting of 1,3,7-trimethy-xantine, 1,3-dimethyl-xanthine and a 3,7-dimethyl-xanthine in a form that is suitable to be used externally and to be absorbed from skin.

17. A weight reduction and inhibition of hypertriglyceridemia composition comprising an effective amount of cyclic AMP (cAMP) or physiologically acceptable salt thereof or both in a form that is suitable to be used nasally and to be absorbed from nasal mucous membrane.

18. The weight reduction and inhibition of hypertriglyceridemia composition comprising an effective amount of cyclic AMP (cAMP) or physiologically acceptable salt thereof or both and an effective amount of at least one inhibitor selected from the group consisting of 1,3,7-trimethy-xantine, 1,3-dimethyl-xanthine and a 3,7-dimethyl-xanthine as defined claim 17, wherein said the weight reduction and inhibition of hypertriglyceridemia composition comprising an effective amount of cyclic AMP (cAMP) or physiologically acceptable salt thereof or both and an effective amount of at least one inhibitor selected from the group consisting of 1,3,7-trimethy-xantine, 1,3-dimethyl-xanthine and a 3,7-dimethyl-xanthinein a form that is suitable to be used nasally and to be absorbed from nasal mucous membrane.

19. A weight reduction and inhibition of hypertriglyceridemia composition comprising an effective amount of cyclic AMP (cAMP) or physiologically acceptable salt thereof or both in a form that is suitable to be injected and to be absorbed intramuscularly.

20. The weight reduction and inhibition of hypertriglyceridemia composition comprising an effective amount of cyclic AMP (cAMP) or physiologically acceptable salt thereof or both and an effective amount of at least one inhibitor selected from the group consisting of 1,3,7-trimethy-xantine, 1,3-dimethyl-xanthine and a 3,7-dimethyl-xanthine as defined claim 19, wherein said the weight reduction and inhibition of hypertriglyceridemia composition comprising an effective amount of cyclic AMP (cAMP) or physiologically acceptable salt thereof or both and an effective amount of at least one inhibitor selected from the group consisting of 1,3,7-trimethy-xantine, 1,3-dimethyl-xanthine and a 3,7-dimethyl-xanthine in a form that is suitable to be injected and to be absorbed intramuscularly.

21. A weight reduction and inhibition of hypertriglyceridemia composition comprising an effective amount of cyclic AMP (cAMP) or physiologically acceptable salt thereof or both in a form that is suitable to be injected and to be absorbed intravenously.

22. The weight reduction and inhibition of hypertriglyceridemia composition comprising an effective amount of cyclic AMP (cAMP) or physiologically acceptable salt thereof or both and an effective amount of at least one inhibitor selected from the group consisting of 1,3,7-trimethy-xantine, 1,3-dimethyl-xanthine and a 3,7-dimethyl-xanthine as defined claim 21, wherein said the weight reduction and inhibition of hypertriglyceridemia composition comprising an effective amount of cyclic AMP (cAMP) or physiologically acceptable salt thereof or both and an effective amount of at least one inhibitor selected from the group consisting of 1,3,7-trimethy-xantine, 1,3-dimethyl-xanthine and a 3,7-dimethyl-xanthine in a form that is suitable to be injected and to be absorbed intravenously.

23. A weight reduction and inhibition of hypertriglyceridemia composition comprising an effective amount of cyclic AMP (cAMP) or physiologically acceptable salt thereof or both in a form that is suitable to be used rectally and to be absorbed from lower segment of intestinal tract.

24. The weight reduction and inhibition of hypertriglyceridemia composition comprising an effective amount of cyclic AMP (cAMP) or physiologically acceptable salt thereof or both and an effective amount of at least one inhibitor selected from the group consisting of 1,3,7-trimethy-xantine, 1,3-dimethyl-xanthine and a 3,7-dimethyl-xanthine as defined claim 23, wherein said the weight reduction and inhibition of hypertriglyceridemia composition comprising an effective amount of cyclic AMP (cAMP) or physiologically acceptable salt thereof or both and an effective amount of at least one inhibitor selected from the group consisting of 1,3,7-trimethy-xantine, 1,3-dimethyl-xanthine and a 3,7-dimethyl-xanthine in a form that is suitable to be used rectally and to be absorbed from lower segment of intestinal tract.
